(19) United States
(12) Patent Application Publication
(10) Pub. No.: US 2026/0358242 A1
(43) Pub. Date: Nov. 12, 2026

(54) GAS SENSOR MODULE AND DEVICE WITH GAS DETECTION FUNCTION

(71) Applicant: Asahi Kasei Microdevices Corporation, Chiyoda-ku, Tokyo (JP)

(72) Inventors: Masanori MASUDA, Chiyoda-ku, Tokyo (JP); Takaaki FURUYA, Chiyoda-ku, Tokyo (JP)

(73) Assignee: Asahi Kasei Microdevices Corporation, Chiyoda-ku, Tokyo (JP)

(21) Appl. No.: 18/599,197

(22) Filed: Mar. 8, 2024

(30) Foreign Application Priority Data
Mar. 24, 2023 (JP) 2023-049046
Jan. 25, 2024 (JP) 2024-009583

(51) Int. Cl. G01N 21/3504; G01N 33/00

(52) U.S. Cl. CPC G01N 21/3504; G01N 33/004

(57) ABSTRACT

A gas sensor module (20) includes a gas sensor (10) and a substrate (21). The gas sensor includes a frame (14), a support (13) supporting the frame (14), and a hollow part (15) that is surrounded by the frame and the support and into which ambient air is introduced, and the gas sensor detects a gas to be detected contained in the ambient air introduced into the hollow part. The substrate is connected to the support. The substrate has a bending stiffness evaluation value, obtained by multiplying a first ratio by a second ratio, of 2 or more, the first ratio being a ratio of a thickness of the gas sensor to a thickness of the substrate, the second ratio being a ratio of Young's modulus of the gas sensor to Young's modulus of the substrate.

### CROSS-REFERENCE TO RELATED APPLICATIONS

The present application claims priority to Japanese Patent Application No. 2023-049046 filed on Mar. 24, 2023, and Japanese Patent Application No. 2024-009583 filed on Jan. 25, 2024, the entire contents of which are incorporated herein by reference.

### TECHNICAL FIELD

The present disclosure relates to a gas sensor module and to a device with a gas detection function.

### BACKGROUND

Gas sensors for detecting gases are used in various fields. Gas sensors are becoming increasingly smaller with the progress of semiconductor and MEMS technologies and are now being mounted inside the housing of various electronic devices. For example, Patent Literature (PTL) 1 discloses an information display apparatus in which a gas sensor is incorporated in a hole that penetrates from the front to the back in a light-emitting panel.

### CITATION LIST

### Patent Literature

PTL 1: JP 2005-227002 A

### SUMMARY

Demand exists for mounting of gas sensors in battery-powered small electronic devices such as smartphones, for example. When a gas sensor is built into a small electronic device, however, an impact (external force) to the housing due to dropping, collision, or the like can transmit vibration to the gas sensor, which can subject the gas sensor to stress. When a gas sensor is subjected to stress, the output characteristics fluctuate. Technology to improve the vibration resistance of gas sensors is needed to prevent the measurement accuracy from being degraded by vibration.

It would be helpful to provide a gas sensor module and a device with a gas detection function that have improved vibration resistance.

(1) A gas sensor module according to an embodiment of the present disclosure includes:
a gas sensor including a frame, a support supporting the frame, and a hollow part that is surrounded by the frame and the support and into which ambient air is introduced, the gas sensor being configured to detect a gas to be detected contained in the ambient air introduced into the hollow part; and
a substrate connected to the support, wherein
the substrate has a bending stiffness evaluation value, obtained by multiplying a first ratio by a second ratio, of 2 or more, the first ratio being a ratio of a thickness of the gas sensor to a thickness of the substrate, the second ratio being a ratio of Young's modulus of the gas sensor to Young's modulus of the substrate.

(2) As an embodiment of the present disclosure, in (1), the first ratio is 2 or more.

(3) As an embodiment of the present disclosure, in (1) or (2), the second ratio is 2 or more.

(4) As an embodiment of the present disclosure, in any one of (1) to (3), the substrate includes polyimide and copper.

(5) As an embodiment of the present disclosure, in any one of (1) to (4), the gas sensor includes a light-emitting element and a detection element that is configured to detect a signal based on light that is emitted from the light-emitting element and passes through the hollow part.

(6) As an embodiment of the present disclosure, in (5), the detection element is a light-receiving element, and the gas sensor is a non-dispersive infrared gas sensor.

(7) As an embodiment of the present disclosure, in (5) or (6), the gas to be detected is carbon dioxide.

(8) A device with a gas detection function according to an embodiment of the present disclosure includes the gas sensor module of (1) or (2).

(9) As an embodiment of the present disclosure, in (8), the device includes a housing, wherein
the gas sensor module is arranged inside the housing.

(10) As an embodiment of the present disclosure, in (8), the device includes a rigid part connected to the substrate, wherein
at least some of a portion of the substrate overlapping with the gas sensor in plan view is connected to the gas sensor,
the rigid part has a higher Young's modulus than the substrate, and
at least some of a portion of the rigid part overlapping with the gas sensor in plan view is connected to the substrate so that a gap is formed between the rigid part and the substrate.

(11) As an embodiment of the present disclosure, in (10), all of the portion of the rigid part overlapping with the gas sensor in plan view is connected to the substrate so that a gap is formed between the rigid part and the substrate.

(12) As an embodiment of the present disclosure, in (10), with respect to a center of the gas sensor in plan view, a fixed point between the substrate and the rigid part is located farther outward than a fixed point between the gas sensor and the substrate.

(13) As an embodiment of the present disclosure, in (10), the rigid part is another substrate different from the substrate.

(14) As an embodiment of the present disclosure, in (9), the device further includes a measurement apparatus configured to detect any one of temperature, humidity, atmospheric pressure, or particles included in the ambient air.

(15) As an embodiment of the present disclosure, in (9), the device further includes a battery.

(16) As an embodiment of the present disclosure, in (10), a ratio zb/zh of acoustic characteristic impedance of the substrate and the rigid part is 0.6 or less, zb being the acoustic characteristic impedance of the substrate, and zh being the acoustic characteristic impedance of the rigid part.

(17) As an embodiment of the present disclosure, in (16), the ratio zb/zh of the acoustic characteristic impedance of the substrate and the rigid part is 0.15 or less.

According to embodiments of the present disclosure, a gas sensor module and a device with a gas detection function that have improved vibration resistance can be provided.

### BRIEF DESCRIPTION OF THE DRAWINGS

In the accompanying drawings:

FIG. 1 is a schematic diagram illustrating a configuration example of a device with a gas detection function provided with a gas sensor module according to an embodiment of the present disclosure;

FIG. 2 is a diagram illustrating a gas sensor;

FIG. 3 is a diagram illustrating a spring equivalent model of the device with a gas detection function in FIG. 1;

FIG. 4 is a diagram illustrating calculated values of the elongation of a gas sensor relative to the elongation of a substrate; and

FIG. 5 is a diagram illustrating the relationship between the acoustic characteristic impedance ratio and energy reflectance.

### DETAILED DESCRIPTION

Embodiments of the present disclosure will be described below with reference to the drawings. In the following description of the drawings, identical or similar parts are labeled with identical or similar symbols. However, the drawings are schematic. For example, the relationship between thickness and width differs from actual dimensions. The embodiments illustrated below are examples of apparatuses to embody the technical ideas of the present disclosure. The shape, structure, arrangement, and the like of constituent components are not limited to the examples below.

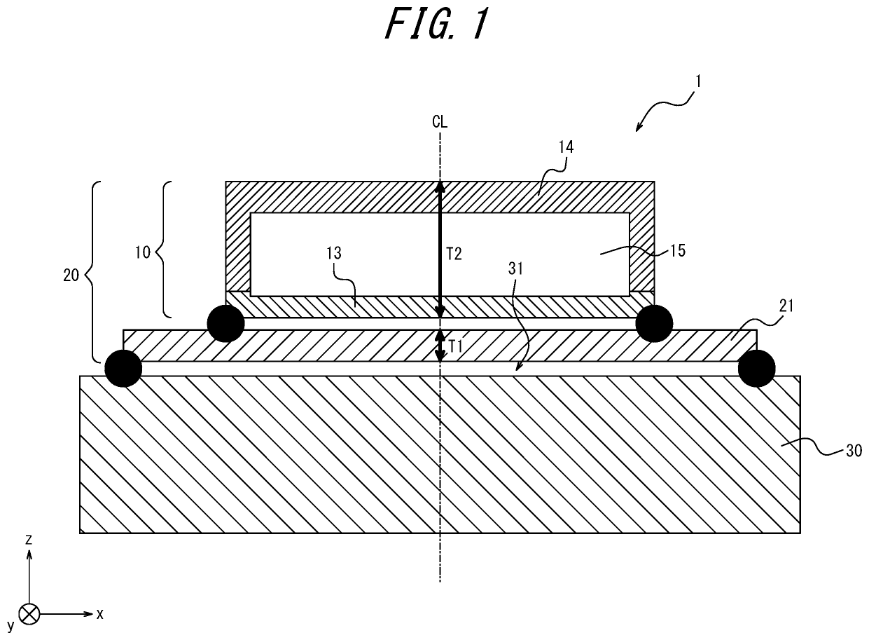

FIG. 1 is a diagram illustrating a configuration of a device 1 with a gas detection function (also referred to simply as “device 1”) according to the present embodiment. The gas detection function is, for example, a function to detect the concentration in the ambient air of a gas to be detected, which is the target of detection. The device 1 includes a housing and a gas sensor module that is arranged inside the housing. The housing is the exterior of the device 1. The gas sensor module 20 includes a gas sensor 10 and a substrate 21. The substrate 21 is connected to a rigid part 30, but the rigid part 30 may be the housing.

In the present embodiment, the device 1 is a small, battery-powered electronic device. In other words, in the present embodiment, the device 1 is also provided with a battery. The device 1 need not be a dedicated gas detection device. In other words, the device 1 may be any of a variety of apparatuses that can perform gas detection as an additional function. Specific examples of the device 1 include a smartphone, a tablet, and a laptop computer. The gas sensor 10 can be built into even a small electronic device such as a smartphone, for example, since the gas sensor 10 has a volume of 1000 mm³ or less and has a combined thickness (height), together with the substrate 21, of 8 mm or less. Since the gas sensor module 20 is sufficiently small, the device 1 may also be further provided with other measurement apparatuses in addition to the gas sensor module 20. The other measurement apparatuses may, for example, be apparatuses that detect temperature, humidity, air pressure, or particles in the ambient air.

The gas sensor module 20 according to the present embodiment includes the gas sensor 10 and the substrate 21, as described above. The gas sensor 10 includes a frame 14, a support 13, and a hollow part 15. The frame 14 is a member that forms the frame portion of the gas sensor 10. The support 13 is a member that supports the frame 14. The hollow part 15 is a space surrounded by the frame 14 and the support 13. Ambient air is introduced into the hollow part 15. At least one of the frame 14 and the support 13 may have holes for ambient air to pass through. The gas sensor 10 detects the gas to be detected contained in the ambient air introduced into the hollow part 15. The substrate 21 is connected to the support 13 of the gas sensor 10. Here, the connection between the substrate 21 and the gas sensor 10 may include an electrical connection, and in the present embodiment the connection includes an electrical connection and a connection that defines (fixes) the position of the gas sensor 10 relative to the substrate 21. The connected, fixed portion is defined as a fixed point.

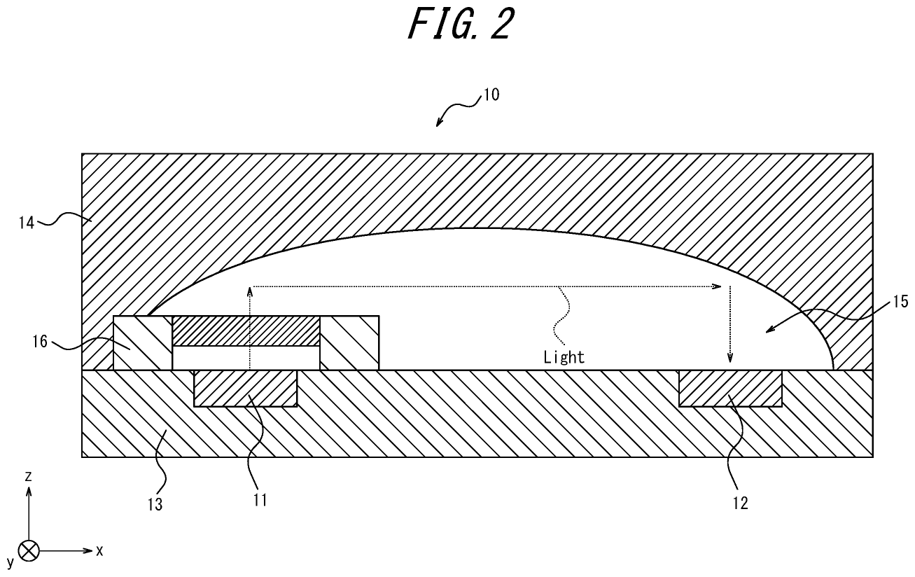

Here, Cartesian coordinates common to FIG. 2 are illustrated in FIG. 1. The z-axis direction is the direction in which the gas sensor 10 and the substrate 21 are stacked and can be referred to as the stacking direction. The z-axis direction is also the thickness direction (height direction) of the gas sensor 10. The x-axis direction corresponds to the width direction of the gas sensor 10. The y-axis direction corresponds to the depth direction of the gas sensor 10. Relative positional relationships may be described hereinafter by designating location in the positive direction of the z-axis as “above” and location in the negative direction of the z-axis as “below”. For example, in the positional relationships among the gas sensor 10, the substrate 21, and the rigid part 30 in FIG. 1, the gas sensor 10 is above the substrate 21, and the rigid part 30 is below the substrate 21. The view of the xy-plane from the front may be referred to below as a plan view. For example, the view when looking in the negative direction of the z-axis and describing the state of the main surface of the substrate 21 through the gas sensor 10 is a plan view. Here, the main surface is the surface with the largest area.

Referring now to FIG. 2, details of the configuration of the gas sensor 10 will be described. In the present embodiment, the gas sensor 10 includes a light-emitting element 11 and a detection element 12 that detects signals based on light that is emitted from the light-emitting element 11 and passes through the hollow part 15. More specifically, the detection element 12 is a light-receiving element, and the gas sensor 10 uses the NDIR (Non Dispersive InfraRed) system. The NDIR system utilizes the fact that the wavelength of infrared radiation that is absorbed differs depending on the type of gas and measures the gas concentration of a gas to be detected by detecting the amount of absorption. The detected gas is carbon dioxide (CO₂) in the present embodiment but is not limited to this example. Other examples include flammable gases such as alcohol (ethanol, etc.), methane, propane, hydrogen, ethylene, and MCH (methylcyclohexane), etc. Other examples include toxic gases such as carbon monoxide, hydrogen sulfide, formaldehyde, and ammonia. Furthermore, Other examples include greenhouse gases such as dinitrogen monoxide and refrigerant gas used in air conditioners, refrigerators, and the like. In the present embodiment, the infrared radiation after being absorbed by the gas to be detected (CO₂) corresponds to the signal based on light emitted from the light-emitting element 11 and passing through the hollow part 15.

The light-emitting element 11 outputs light containing a wavelength that is absorbed by the gas to be measured. Specific examples of the light-emitting element 11 are a Light Emitting Diode (LED) and a Micro Electro Mechanical Systems (MEMS) light source. In the present embodiment, the light-emitting element 11 is an infrared LED. The wavelength of the infrared light in the present embodiment is 4.3 μm, which is the absorption band of CO₂, but the wavelength may be selected in the range of 2 μm to 12 μm, for example, depending on the gas to be detected.

The light-receiving element is sensitive to a band of light that includes wavelengths absorbed by the gas to be measured. As a specific example, the light-receiving element may be a quantum-type sensor, such as a photodiode with a PIN structure. In the present embodiment, the light-receiving element is a quantum-type infrared sensor.

A reflector may be provided on the side surface of the hollow part 15 of the frame 14 so that light emitted from the light-emitting element 11 is reflected and incident on the light-receiving element. The reflector functions as a light-guiding portion to guide light from the light-emitting element 11 to the light-receiving element. The reflector may, for example, be a concave mirror with a reflective surface formed by a metal having a high reflectivity, such as aluminum or gold.

The gas sensor 10 may include a filter 16 that limits the wavelength of light. In the example in FIG. 2, the filter 16 is provided on the light-emitting element 11, but it suffices for the filter 16 to be in the optical path, for example on at least one of the light-emitting element 11, the light-receiving element, and the optical guide.

The support 13 may be a molded resin that supports the frame 14 and seals the base portion of the filter 16, the light-emitting element 11, and the light-receiving element 12. The support 13 may be a general printed circuit board to which the light-emitting element 11 and the light-receiving element 12 are connected. The support 13 may have electrical wiring running through it. The support 13 may also seal or connect an apparatus such as a calculation apparatus that calculates the gas concentration based on the detection signal outputted from the light-receiving element.

Referring again to FIG. 1, the gas sensor module 20 is housed in a housing and is connected to the rigid part 30, which is a member of the device 1. The rigid part 30 is, for example, the housing of the device 1 or another substrate different from the substrate 21. Here, if an external force is applied to the housing, for example due to dropping, collision, or the like of the device 1, the vibration is transmitted to the gas sensor 10 via the rigid part 30 and can become a stress on the gas sensor 10. Stress on the gas sensor 10 causes fluctuations in the output characteristics and reduces the measurement accuracy. Upon diligently investigating ways to reduce vibration in the gas sensor 10, we discovered that vibration is less likely to be transmitted to the gas sensor 10 when the thickness and Young's modulus of the substrate 21 have a predetermined relationship to the gas sensor 10, as described below.

Specifically, we modeled the gas sensor 10, the substrate 21, and the rigid part 30 and verified by simulation that the strain on the gas sensor 10 varies depending on the ratio of the cross-sectional area and elastic modulus of the gas sensor 10 and the substrate 21. The cross-sectional area may be defined as the area of a cross-section cut by a virtual plane (CL) parallel to the yz-plane illustrated in FIG. 1. CL may be located at the intermediate position of the width of the gas sensor 10 in the x-axis direction, i.e., the midpoint in the width direction of the gas sensor 10. Simulations indicted that the vibration transmitted to the gas sensor 10 tends to be smaller when the cross-sectional area of the substrate 21 is small relative to the cross-sectional area of the gas sensor 10. Simulations also indicted that the vibration transmitted to the gas sensor 10 tends to be smaller when the elastic modulus of the substrate 21 is small relative to the elastic modulus of the gas sensor 10, i.e., when the substrate 21 deforms relatively easily. This is thought to be attributable to vibration propagating to areas where vibration is more easily propagated.

Here, in the gas sensor module 20, the length in the depth direction (length in the y-axis direction) is often almost the same for the gas sensor 10 and the substrate 21 from the perspective of miniaturization. The ratio of the cross-sectional areas of the gas sensor 10 and the substrate 21 can therefore be replaced by the ratio of their thicknesses. Furthermore, the elastic modulus can be indicated by Young's modulus, since the bending stiffness mainly affects the vibration. A bending stiffness evaluation value for the substrate 21 is therefore introduced so that the ratio of the cross-sectional area and elastic modulus of the substrate 21 to those of the gas sensor 10 can be illustrated numerically. The bending stiffness evaluation value (EV) is obtained by multiplying a first ratio (R1) of the thickness (Ts) of the gas sensor 10 to the thickness (Tb) of the substrate 21 by a second ratio (R2) of Young's modulus (Es) of the gas sensor 10 to Young's modulus (Eb) of the substrate 21, and by a third ratio (R3) of the width (Ws) of the gas sensor 10 to the width (Wb) of the substrate 21. In other words, the bending stiffness evaluation value is calculated as EV=R1×R2×R3=(Ts/Tb)×(Es/Eb)×(Ws/Wb). In other words, EV is the hardness ratio between the substrate 21 and the gas sensor 10, and a larger value of EV indicates that the substrate 21 is softer relative to the gas sensor 10. Here, the width is the length in the paper depth direction in FIG. 1.

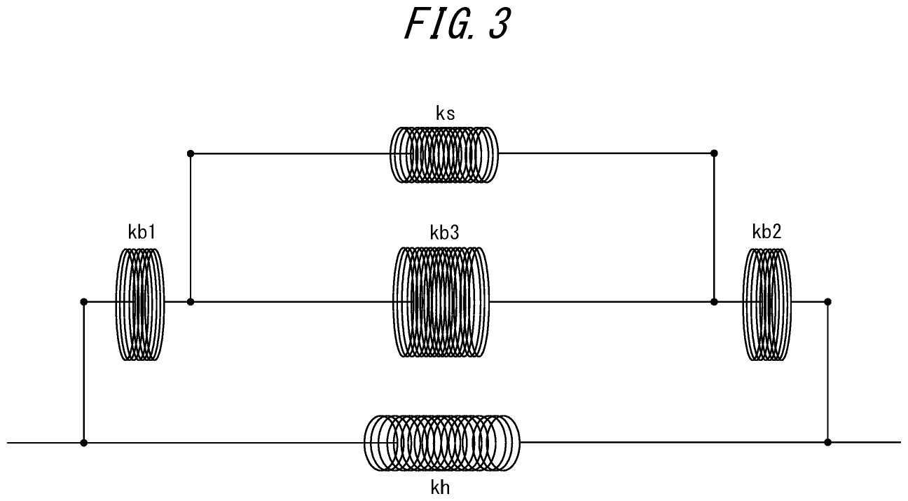

With respect to the aforementioned bending stiffness evaluation value, FIG. 3 illustrates a spring equivalent model of the device 1 in FIG. 1. The spring equivalent model is explained in detail below with reference to FIG. 3. The spring kh models a portion of the rigid part 30 between two connected portions of the rigid part 30 and the substrate 21. The spring ks models the gas sensor 10. The springs kb1 and kb2 model respective left and right portions of the substrate 21 between the portion connected to the rigid part 30 and the portion connected to the gas sensor 10. The spring kb3 models the portion of the substrate 21 between the two connected portions of the gas sensor 10 and the substrate 21.

When the rigid part 30 is deformed by pressure, impact, sound, or the like, the spring kh expands and contracts, and the displacement of the spring kh at this time is defined as a displacement X. In this case, the coupled spring K consisting of the spring ks, the spring kb1, the spring kb2, and the spring kb3 expands and contracts by the same displacement X. The displacement by which each spring in the coupled spring K expands and contracts is determined by the stiffness of each spring. The displacement of the gas sensor is defined here as xs, the spring constant of the spring ks as kₖₛ, the spring constant of spring kb1 as k_{kb1}, the spring constant of spring kb2 as k_{kb2}, the spring constant of spring kb3 as k_{kb3}, and the spring constant of the coupled spring K as kk. Assuming that the rigid part 30 is subjected to an impact force F due to pressure, impact, sound, or the like, the forces on the springs are balanced. The force on the coupled spring K and the force on the gas sensor 10 are therefore equivalent. This yields the following Equation (a), which can be expressed as Equation (b).

$$F = ( {k}_{ks} + {k}_{kb 3} ) xs = {k}_{K} X \qquad Equation ( a )$$

$$\frac{xs}{X} = \frac{{k}_{K}}{{k}_{ks} + {k}_{kb 3}} \qquad Equation ( b )$$

Here, xs/X corresponds to the elongation of the gas sensor relative to the elongation of the substrate. As a simple system, the gas sensor is fixed in the center of a substrate that is three times the length of the gas sensor, i.e., Lb=3Ls, and a system such that k_{kb1}=k_{kb2}=k_{kb} is considered below. Assuming that the material of the substrate is sufficiently soft (kₖₛ>>k_{kb3}) relative to the material of the gas sensor, the system can be expressed as Equation (c).

$$\frac{xs}{X} \approx \frac{{k}_{K}}{{k}_{ks}} \qquad Equation ( c )$$

Furthermore, the coupled spring K becomes the following Equation (d), which can be expressed as Equation (e).

$$\frac{1}{{k}_{K}} = \frac{1}{{k}_{kb 1}} + \frac{1}{{k}_{kb 2}} + \frac{1}{{k}_{ks} + {k}_{kb 3}} \approx \frac{2}{{k}_{kb}} + \frac{1}{{k}_{ks}} \qquad Equation ( d )$$

$${k}_{K} = \frac{{k}_{kb} {k}_{ks}}{2 {k}_{ks} + {k}_{kb}} \qquad Equation ( e )$$

Consequently, the following Equation (f) holds.

$$\frac{xs}{X} \approx \frac{{k}_{kb}}{2 {k}_{ks} + {k}_{kb}} = \frac{1}{2 ( \frac{{k}_{ks}}{{k}_{kb}} + 1 )} \qquad Equation ( f )$$

The spring constant of each spring can be obtained by dividing the product of the cross-sectional area and Young's modulus by the length of the member being modeled as each spring. In other words, this yields the following Equation (g), which can be expressed as Equation (h).

$${k}_{s} = \frac{EsTsWs}{Ls} \qquad Equation ( g )$$

$${k}_{kb} = \frac{EbTbWb}{\frac{Lb - Ls}{2}} = \frac{2 EbTbWb}{Lb - Ls} \qquad Equation ( h )$$

Furthermore, the bending stiffness evaluation value EV can be used to write the following Equation (i), which becomes Equation (j).

$$\frac{{k}_{ks}}{{k}_{kb}} = \frac{Es}{Eb} \frac{Ts}{Tb} \frac{Ws}{Wb} \frac{Lb - Ls}{2 Ls} = α R 1 R 2 R 3 = α EV \qquad Equation ( i )$$

$$\frac{xs}{X} = \frac{1}{2 EV + 1} \qquad Equation ( j )$$

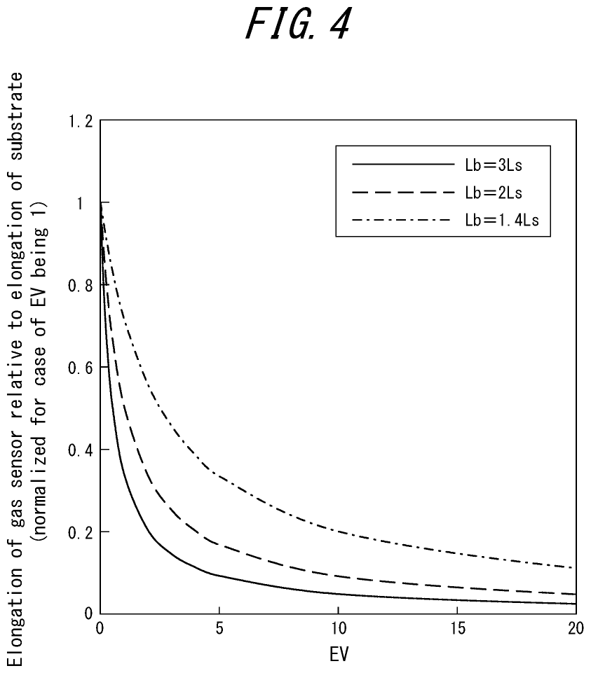

In other words, the larger the bending stiffness evaluation value EV and the softer the substrate 21 in relation to the gas sensor 10, the smaller the displacement and vibration transmitted to the gas sensor 10. For example, in a case in which the springs kb1 and kb2 are sufficiently softer than the spring ks, EV will be larger and the value of xs/X will be smaller. In this case, the total displacement of the springs kb1 and kb2 becomes almost equal to the displacement X, and the displacement of springs ks and kb3 becomes approximately zero. In other words, a configuration that does not displace the gas sensor 10 can be adopted, achieving the effect of suppressing vibration. That is, the portions corresponding to the springs kb1 and kb2 are important in achieving the effect of suppressing vibration. In order to achieve such a structure, the connection between the substrate 21 and the rigid part 30 needs to be located outward from the connection between the gas sensor 10 and the substrate 21 in a top view. In other words, the fixed point between the substrate 21 and the rigid part 30 needs to be located outward from the fixed point between the gas sensor 10 and the substrate 21 with respect to the center of the gas sensor 10 in plan view. FIG. 4 illustrates some calculated values of the elongation of the gas sensor 10 relative to the elongation (displacement) of the substrate 21.

Here, the thickness of the gas sensor 10 and the substrate 21 may be measured at CL in FIG. 1, i.e., at the midpoint in the width direction in the gas sensor 10, but the measurement position is not limited. In particular, FIG. 1 illustrates one example of measuring the thickness of the substrate 21, but as another example, the thickness may be measured at the portion corresponding to the springs kb1 and kb2. In a case in which the substrate 21 is not of uniform thickness, the value of the thinnest portion may be used as the thickness of the substrate 21. The Young's modulus of the gas sensor 10 and the substrate 21 may be measured by known methods such as experimentation. In this case, the value of Young's modulus at CL in FIG. 1, i.e., at the midpoint in the width direction, may be used as a representative value. However, as with the above-described thickness, Young's modulus is not limited to the value at the midpoint in the width direction, and values at positions other than the midpoint in the width direction may also be used as representative values. As another method, the Young's modulus of the substrate 21 may be calculated from the material of the substrate 21 or the like. The Young's modulus of the gas sensor 10 may also be determined by calculation from the material of the support 13, treating the support 13 as a major part.

If the bending stiffness evaluation value is equal to or greater than a predetermined value for the substrate 21, the vibration transmitted to the gas sensor 10 can be reduced. In other words, a substrate 21 with a bending stiffness evaluation value of 2 or more is used in the gas sensor module 20. It suffices for the bending stiffness evaluation value to be 2 or more, but as an example, the bending stiffness evaluation value is 2 or is a positive number greater than 2. For example, in a case in which the first ratio (R1=Ts/Tb) is 2 or more, the thickness of the substrate 21 is half or less that of the gas sensor 10. This is considered to sufficiently achieve the effect of reducing vibration transmission. Furthermore, in a case in which the second ratio (R2=Es/Eb) is 2 or more, for example, the Young's modulus of the substrate 21 is half or less that of the gas sensor 10. This is considered to sufficiently achieve the effect of reducing vibration transmission. It is not always necessary to set a threshold (2 in the above example) for the first ratio, the second ratio, and the third ratio. That is, if the first ratio is sufficiently large, then even if the second ratio or third ratio is close to 1, for example, the effect of reducing the transmission of vibration can be achieved. Also, if the second ratio is sufficiently large, then even if the first ratio or third ratio is close to 1, the effect of reducing the transmission of vibration can be achieved. It thus suffices for the bending stiffness evaluation value obtained by multiplying the first ratio, the second ratio, and the third ratio to be equal to or greater than a predetermined value. While it suffices for the predetermined value of the bending stiffness evaluation value to be 2 or more, 10 or more is preferable. The predetermined value of the bending stiffness evaluation value is more preferably 100 or more, even more preferably 1×10³ or more. The predetermined value of the bending stiffness evaluation value is still more preferably 1×10⁴ or more, and yet more preferably 1×10⁵ or more. If the bending stiffness evaluation value is too large, i.e., if the substrate is extremely soft compared to the gas sensor 10, the substrate will be greatly distorted, and the connection between the substrate 21 and the gas sensor 10 will be weak. As a result, the connected portion may be damaged in the event of an impact. From the viewpoint of preventing damage, the bending stiffness evaluation value is preferably 1×10⁷ or less, more preferably 1×10⁶ or less.

The vibration transmitted to the gas sensor 10 is generated or transmitted in the rigid part 30, which is the housing or is another substrate, as impact, sound, or the like generated by dropping or carrying, for example. This vibration is transmitted to the gas sensor 10 via the substrate 21. In other words, if the vibration is not transmitted from the rigid part 30 to the substrate 21, the vibration reaching the gas sensor 10 can be reduced. The reduction of vibration is achieved by the large difference in acoustic characteristic impedance between the rigid part 30 and the substrate 21. That is, in a case in which the difference in acoustic characteristic impedance between the rigid part 30 and the substrate 21 is large, vibration is reflected at the connection between the rigid part 30 and the substrate 21, and penetration of vibration into the substrate 21 is suppressed. Letting the density of the rigid part 30 be ρh, the sound velocity of the rigid part 30 be ch, the density of the substrate 21 be ρb, and the sound velocity of the substrate 21 be cb, the acoustic characteristic impedance zh of the rigid part 30 is zh=ρh×ch. The acoustic characteristic impedance zb of the substrate 21 is zb=ρb×cb. The energy reflectance R at the connection between the rigid part 30 and the substrate 21 can be expressed by the following Equation (1).

$$R = {( \frac{zh - zb}{zh + zb} )}^{2} = {( \frac{1 - \frac{zb}{zh}}{1 + \frac{zb}{zh}} )}^{2} \qquad Equation ( 1 )$$

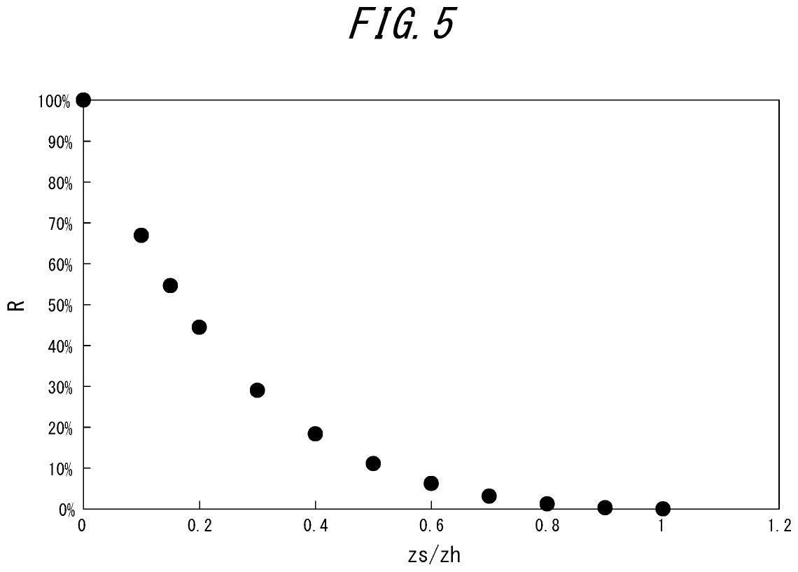

FIG. 5 illustrates the numerical calculation results of the energy reflectance R. The energy reflectance R begins to rise when the ratio of the acoustic characteristic impedance between the substrate 21 and the rigid part 30 (zb/zh) is 0.6 or less, and the energy reflectance R exceeds 50% when (zb/zh) is approximately 0.15 or less, which can achieve the effect of reducing the transmission of vibration. Here, the sound velocity cᵢ in each medium i is given by the following Equation (2), using Young's modulus Eᵢ, the density ρᵢ, and Poisson's ratio μᵢ for medium i.

$${c}_{i} = \sqrt{\frac{Ei}{ρi} \cdot \frac{1 - {μ}_{i}}{( 1 + {μ}_{i} ) ( 1 - 2 {μ}_{i} )}} \qquad Equation ( 2 )$$

For example, when the rigid part 30 is aluminum, which is a soft metal, and the substrate 21 is a flexible substrate, the Young's modulus of aluminum is 75.5 GPa. The Young's modulus of polyimide, which is a material for flexible substrates, is 5 GPa. The ratio (zb/zh) of the aforementioned acoustic characteristic impedance is approximately 0.066. The energy reflection coefficient R is 76.7%, and a large vibration suppression effect is achieved. FIG. 5 illustrates the relationship between the acoustic characteristic impedance ratio (zb/zh) and the energy reflectance R.

Here, as a configuration to achieve a sufficiently small bending stiffness evaluation value, the substrate 21 may be a flexible substrate. The flexible substrate is thin and flexible and includes polyimide and copper, for example. The substrate 21 is connected to the rigid part 30, and the rigid part 30 is, for example, a housing or another substrate and has at least a higher Young's modulus than the substrate 21. If the substrate 21 is fixed on the rigid part 30 without any space therebetween, the flexibility provided by the substrate 21 is not utilized, and the vibration caused by external forces is directly transmitted to the gas sensor 10. Therefore, as illustrated in FIG. 1, the substrate 21 is connected to the rigid part 30 via the fixed points so that a gap 31 is created (space is provided). In the present disclosure, the gap 31 is defined as a portion that is not mechanically connected. For example, a portion that is in contact but not physically fixed is included in the definition of the gap 31. In the example in FIG. 1, the substrate 21 is connected to the rigid part 30 via fixed points so that all of the portion overlapping the gas sensor 10 in plan view has the gap 31 between the substrate 21 and the rigid part 30. Here, it suffices for the substrate 21 to be connected to the rigid part 30 via the fixed points so that at least some, rather than all, of the portion of the substrate 21 overlapping with the gas sensor 10 in plan view has the gap 31 between the substrate 21 and the rigid part 30. The fixed points connecting the substrate 21 and the rigid part 30 may be bonding with solder, adhesive, or the like, or may be removable connections using socket pins, screws, magnets, or the like.

The gas sensor module 20 and the device 1 with a gas detection function according to the present embodiment can reduce the vibration transmitted to the gas sensor 10 and improve vibration resistance with the above configuration. Therefore, the gas sensor module 20 and the device 1 with a gas detection function according to the present embodiment are particularly suitable for installation in small battery-powered electronic devices that are portable (not installed in a fixed location).

Although embodiments have been described based on the various drawings and examples, it should be noted that a person of ordinary skill in the art could easily make various modifications and revisions based on the present disclosure. Accordingly, such modifications and revisions should also be considered to be included within the scope of the present disclosure.

In the above embodiments, the gas sensor 10 has been described as being an NDIR gas sensor and the detection element 12 as being a light-receiving element, but the gas sensor 10 may have a different configuration. For example, the gas sensor 10 may be a photoacoustic gas sensor. In this case, the detection element 12 is configured by a microphone, for example. In the photoacoustic method, the gas concentration is measured by using a high-performance microphone to pick up, as sound, the vibrations of gas molecules that have absorbed light.

1. A gas sensor module comprising:
a gas sensor comprising a frame, a support supporting the frame, and a hollow part that is surrounded by the frame and the support and into which ambient air is introduced, the gas sensor being configured to detect a gas to be detected contained in the ambient air introduced into the hollow part; and
a substrate connected to the support, wherein
the substrate has a bending stiffness evaluation value, obtained by multiplying a first ratio by a second ratio, of 2 or more, the first ratio being a ratio of a thickness of the gas sensor to a thickness of the substrate, the second ratio being a ratio of Young's modulus of the gas sensor to Young's modulus of the substrate.

2. The gas sensor module according to claim 1, wherein the first ratio is 2 or more.

3. The gas sensor module according to claim 1, wherein the second ratio is 2 or more.

4. The gas sensor module according to claim 1, wherein the substrate comprises polyimide and copper.

5. The gas sensor module according to claim 1, wherein the gas sensor comprises a light-emitting element and a detection element that is configured to detect a signal based on light that is emitted from the light-emitting element and passes through the hollow part.

6. The gas sensor module according to claim 5, wherein the detection element is a light-receiving element, and the gas sensor is a non-dispersive infrared gas sensor.

7. The gas sensor module according to claim 6, wherein the gas to be detected is carbon dioxide.

8. A device with a gas detection function, the device comprising the gas sensor module according to claim 1.

9. The device with a gas detection function according to claim 8, further comprising a housing, wherein
the gas sensor module is arranged inside the housing.

10. The device with a gas detection function according to claim 8, further comprising a rigid part connected to the substrate, wherein
at least some of a portion of the substrate overlapping with the gas sensor in plan view is connected to the gas sensor,
the rigid part has a higher Young's modulus than the substrate, and
at least some of a portion of the rigid part overlapping with the gas sensor in plan view is connected to the substrate so that a gap is formed between the rigid part and the substrate.

11. The device with a gas detection function according to claim 10, wherein all of the portion of the rigid part overlapping with the gas sensor in plan view is connected to the substrate so that a gap is formed between the rigid part and the substrate.

12. The device with a gas detection function according to claim 10, wherein with respect to a center of the gas sensor in plan view, a fixed point between the substrate and the rigid part is located farther outward than a fixed point between the gas sensor and the substrate.

13. The device with a gas detection function according to claim 10, wherein the rigid part is another substrate different from the substrate.

14. The device with a gas detection function according to claim 9, further comprising a measurement apparatus configured to detect any one of temperature, humidity, atmospheric pressure, or particles included in the ambient air.

15. The device with a gas detection function according to claim 9, further comprising a battery.

16. The device with a gas detection function according to claim 10, wherein a ratio zb/zh of acoustic characteristic impedance of the substrate and the rigid part is 0.6 or less, zb being the acoustic characteristic impedance of the substrate, and zh being the acoustic characteristic impedance of the rigid part.

17. The device with a gas detection function according to claim 16, wherein the ratio zb/zh of the acoustic characteristic impedance of the substrate and the rigid part is 0.15 or less.
